(19) United States
(12) Patent Application Publication
(10) Pub. No.: US 2026/0015525 A1
(43) Pub. Date: Jan. 15, 2026

(54) ARTICLE FIXTURE, METHOD FOR REMOVING ARTICLE, AND ADHESIVE SHEET UNIT

(71) Applicant: Solventum Intellectual Properties Company, Maplewood, MN (US)

(72) Inventors: Yasuhiro KINOSHITA, Tokyo (JP); Ryoko KUNIBU, Tokyo (JP); Katsumasa NEMOTO, Tokyo (JP)

(73) Assignee: Solventum Intellectual Properties Company, Maplewood, MN (US)

(21) Appl. No.: 18/993,876

(22) PCT Filed: Jul. 11, 2023
(86) PCT No.: PCT/IB2023/057110

§ 371 Date: Jan. 13, 2025

(30) Foreign Application Priority Data
Jul. 27, 2022 (JP) 2022-119372

(51) Int. Cl. C09J 5/00; C09J 7/24

(52) U.S. Cl. CPC C09J 5/00; C09J 7/241; C09J 2301/124; C09J 2301/308; C09J 2301/502; C09J 2423/006

(57) ABSTRACT

To provide an article fixture, a method for removing an article, and an adhesive sheet unit that can reduce damage to an adherend.

An article fixture for fixing an article on an adherend includes a protective layer that can be formed on the adherend and includes a first main surface facing the adherend and a second main surface opposite to the adherend; and an adhesive sheet unit for bonding to the second main surface of the protective layer. The adhesive sheet unit includes a first adhesive layer for bonding to the second main surface, and the adhesive sheet unit is formed to be stretchable in at least two directions when the article is attached.

### TECHNICAL FIELD

One aspect of the present invention relates to an article fixture, a method for removing an article, and an adhesive sheet unit.

### BACKGROUND ART

With the progress and spread of wearable device technology, there is an increasing opportunity to directly fix various medical devices and health monitoring devices to the skin even in medical practice. For continuous and reliable monitoring and sensing, the device needs to be securely fixed on the skin. A medical tape is generally used for fixing the device. Together with the fixing force, the object is to apply no load to the skin at the time of removal. In particular when the device is integrated on the medical tape, the removal of the device will tear the tape off the skin without any angle. Therefore, the skin is substantially lifted, and trouble such as skin peeling and skin laceration may occur.

Patent Document 1 below discloses an acrylic adhesive medical tape with controlled adhesive force. Patent Document 2 below discloses a silicone adhesive medical tape. These tapes can reduce the risk of occurrence of the above-described trouble, but cannot provide the original purpose of monitoring and sensing in some cases due to insufficient fixing force. In recent years, there have been increasing cases where monitoring and sensing are continuously performed for a long period of one week, two weeks, or more, and it has been required to achieve both a strong fixing force and a reduction in load on the skin at the time of removal.

### SUMMARY

### Technical Problem

Here, when an article is removed from an adherend, it is necessary to peel an article fixture from the adherend. When the article fixture is peeled off in a state where the article is attached, the article fixture is peeled off from the adherend without forming an angle, and thus there is a problem that the adherend is damaged.

An object of one aspect of the present invention is to provide an article fixture, a method for removing an article, and an adhesive sheet unit that can reduce damage to an adherend.

### Solution to Problem

An article fixture according to an embodiment of the present invention is an article fixture for fixing an article on an adherend, the article fixture including: a protective layer configured to be formed on the adherend, the protective layer including a first main surface facing the adherend, and a second main surface opposite to the adherend; and an adhesive sheet unit configured to bond to the second main surface of the protective layer, wherein the adhesive sheet unit includes a first adhesive layer configured to bond to the second main surface, and the adhesive sheet unit is formed to be stretchable in at least two directions when the article is attached.

A method for removing an article according to an embodiment of the present invention is a method for removing an article fixed by the above-described article fixture, the method including detaching the adhesive sheet unit from a protective layer by stretching the adhesive sheet unit in at least two directions with the protective layer left on the adherend.

An adhesive sheet unit according to an embodiment of the present invention is disposed between a protective layer formed on an adherend and an article when the article is fixed on the adherend, the adhesive sheet unit including a first adhesive layer bonded to the protective layer, wherein the adhesive sheet unit is formed to be stretchable in at least two directions when the article is attached.

### Advantageous Effects of Invention

According to an embodiment of the present invention, damage to an adherend can be reduced.

### BRIEF DESCRIPTION OF DRAWINGS

FIG. 1 is a perspective view of an article fixture according to an embodiment.

FIG. 2 is a cross-sectional view of the article fixture.

FIG. 3 is a cross-sectional view of the article fixture.

FIG. 4 is a cross-sectional view of the article fixture.

FIG. 5 is a plan view illustrating an adhesive sheet unit.

FIG. 6 is a view illustrating a procedure of fixing and removing an article by the article fixture.

FIG. 7 is a plan view illustrating the adhesive sheet unit.

FIG. 8 is a plan view illustrating the adhesive sheet unit.

FIG. 9 is a plan view illustrating the adhesive sheet unit.

FIG. 10 is a plan view illustrating the adhesive sheet unit.

FIG. 11 is a plan view illustrating the adhesive sheet unit.

FIG. 12 is a plan view illustrating the adhesive sheet unit.

FIG. 13 is a table showing members that can be selected for testing.

FIG. 14 is a table showing members that can be selected for testing.

FIG. 15 is a view illustrating test results of peel and tensile adhesive forces.

FIG. 16 is a view illustrating test results of peel and tensile adhesive forces.

FIG. 17 is a table illustrating test conditions and test results of Examples and Comparative Examples.

FIG. 18 is a table showing results of a static shear test.

FIG. 19 is a table showing test results of a tensile strength test.

### DESCRIPTION OF EMBODIMENTS

Detailed descriptions of the embodiments according to the present invention are given below with reference to the attached drawings. In the description of the drawings, identical or equivalent elements are denoted by the same reference signs, and redundant description of such elements will be omitted.

An article fixture 1 according to the present embodiment is a member for fixing an article 2 on an adherend 3. The adherend 3 is, for example, human skin. In addition, the adherend 3 may be skin of an animal or the like, but is not limited thereto. The article 2 is various devices to be attached to human skin. As the article 2, various devices such as a continuous blood glucose measurement sensor, an insulin pump, a heart rate sensor, a thermometer, various wearable devices, an IC chip, and an NFC tag are employed.

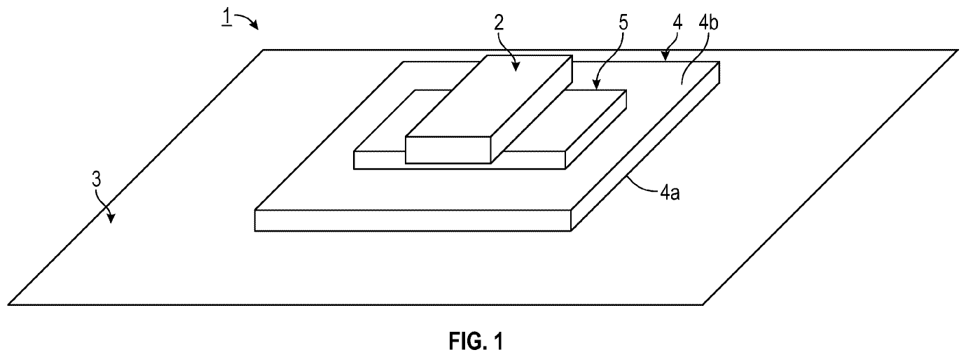

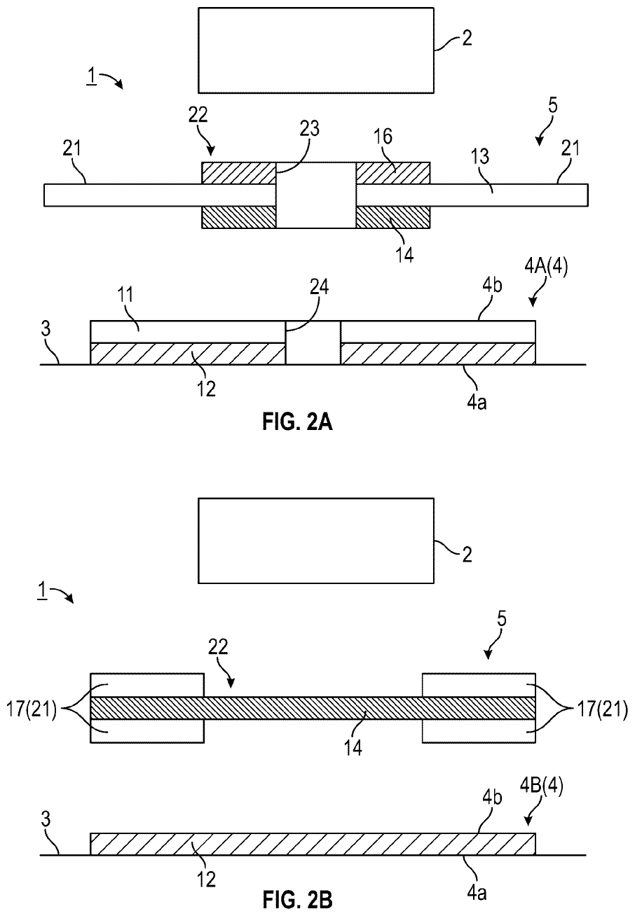

As illustrated in FIG. 1, the article fixture 1 includes a protective layer 4 and an adhesive sheet unit 5. The protective layer 4 is a member formed on the adherend 3. The protective layer 4 can be formed on the adherend 3 and includes a first main surface 4a facing the adherend 3 and a second main surface 4b on the opposite side of the adherend 3. As illustrated in FIG. 2(a), the protective layer 4A includes a protective substrate 11 having the second main surface 4b and a second adhesive layer 12 formed on the first main surface 4a. The protective substrate 11 is a sheet-like member for supporting the second adhesive layer 12 formed on the first main surface 4a side. The second adhesive layer 12 is a member for bonding to the adherend 3.

As illustrated in FIG. 2(b), the protective layer 4B may be formed as a protective coating layer by applying a protection liquid to the adherend 3.

The second adhesive layer 12 of the protective layers 4A and 4B may have a holding force of 50 minutes or more, 200 minutes or more, or 500 minutes or more under a load condition of 30° C., 75% RH, and 300 g with respect to Protein Leather (trade name; IDEATEX JAPAN Co., Ltd).

As the components of the second adhesive layer 12 in the protective layer 4A illustrated in FIG. 2 (a), for example, the following may be employed. For the second adhesive layer 12, typically, adhesives suitable for use in medical applications may be employed. Generally, preferable adhesives for medical use include acrylic pressure sensitive adhesives, rubber-based pressure sensitive adhesives, and silicone- based pressure sensitive adhesives such as those described in PCT WO 03057741, PCT WO 9918166, and PCT WO 0032142. For example, the rubber-based pressure sensitive adhesives may be a mixture of a synthetic rubber such as styrene-isoprene-styrene (SIS) rubber and a tackifier such as a rosin-based tackifier. Examples of other synthetic rubbers further include styrene butadiene styrene (SBS), styrene butadiene rubber (SBR), nitrile butyl rubber (NBR), chloroprene rubber, silicone rubber, acrylic rubber, butyl rubber, urethane rubber, ethylene propylene rubber, and fluororubber.

Another pressure sensitive adhesive that can be employed for the second adhesive layer 12 may be, for example, a copolymer of (i) at least one of monoethylenically unsaturated (meth) acrylates (hereinafter, referred to as “first monomer”) having an alkyl group having at least 4 carbon atoms on average and (ii) at least one of monoethylenically unsaturated reinforcing monomers (hereinafter, referred to as a “second monomer”). In addition, as the second adhesive layer 12, those described in paragraphs 0013 to 0038 of JP 2009-530058 T may be employed.

The protective substrate 11 as illustrated in FIG. 2 (a) may be any substrate that can be used as pressure-sensitive adhesive tapes in medical applications. For example, the protective substrate 11 may be perforated films, open-celled foam sheets, foams or laminated combinations thereof, all of which are made of an organic polymer. Examples of the organic polymer include polyolefins such as polyethylene, polypropylene, polybutene, an ethylene-propylene copolymer, an ethylene-vinyl acetate copolymer, an ethylene-ethyl acrylate copolymer; polyurethanes; polyesters; polyamides; and other plastics. The protective substrate 11 may also be air-permeable and moisture-permeable in itself. For example, the protective substrate 11 may be made of fabrics, nonwoven fabrics, meltblown webs, foams, spunbonded webs, thermally bonded webs, spunlace webs, paper, and thermally embossed nonwoven fabrics, as well as those described in U.S. Pat. No. 5,496,603. More precisely, examples of the protective substrate 11 may be textiles, cotton, knitted or non-woven fabrics of organic polymers such as polyvinyl alcohol or cellulose, paper, and perforated films of polyvinyl alcohol. If desired, the protective substrate 11 may be subjected to a water repellent treatment with a known water repellent agent. The protective substrate 11 may be elastic or inelastic. Preferably, the protective substrate 11 has good air permeability and moisture permeability, and also has good elasticity. Particularly preferred are elastic cotton fabrics (textiles) or nonwoven fabrics. Further, waterproof substrates (for example, urethane films) may be used. The protective substrate 11 may have a thickness of 15 to 2,000 micrometers.

When the protective layer 4B illustrated in FIG. 2 (b) is employed, the protective layer 4B may be formed as follows. The protective layer 4B may be formed through a film-forming step of applying a primer containing a silicone acrylic resin and an alkane solvent to the skin to form a solid film on the skin. As the protective layer 4B, those described in paragraphs 0013 to 0025 of JP 2020-103427 A may be employed.

The film-forming step is a step of forming a solid film on the skin by applying a primer containing a silicone acrylic resin and an alkane solvent to the skin. The silicone acrylic resin may be, for example, a homopolymer of siloxysilane having a (meth) acryloyloxy group, or a copolymer of the siloxysilane and another polymerizable monomer.

Examples of the siloxysilane having a (meth) acryloyloxy group include 3-(meth)acryloyloxypropyl tris (trimethylsiloxy) silane, 3-(meth) acryloy loxypropylpentamethyl disiloxane, 3-(meth) acryloyloxypropyl bis(trimethylsiloxy) methylsilane, and 3-(meth)acryloyloxypropylmethyl bis(trimethylsiloxy) silane. Such siloxysilane may be used alone or in combination of two or more types.

Examples of the polymerizable monomer that can form a copolymer with siloxysilane include methyl (meth)acrylate, tetrahydrofurfuryl (meth)acrylate, cyclohexyl (meth)acrylate, n-lauryl (meth)acrylate, 2-phenoxyethyl (meth)acrylate, isodecyl (meth)acrylate, isooctyl (meth)acrylate, isobornyl (meth)acrylate, benzyl (meth)acrylate, 2-butoxyethyl (meth)acrylate, n-butyl (meth)acrylate, ethyl (meth)acrylate, dimethyl itaconate, di-n-butyl itaconate, 2-ethylhexyl (meth)acrylate, furfuryl (meth)acrylate, n-hexyl (meth)acrylate, isobutyl (meth)acrylate, isopropyl (meth)acrylate, α-methylstyrene, styrene, p-t-butylstyrene, 4-methoxystyrene, n-octadecyl (meth)acrylate, 2-phenylethyl (meth)acrylate, n-tridecyl (meth)acrylate, vinyl benzoate, vinyl naphthalene, fluorinated siloxane, fluorinated itaconate, fluorinated (meth)acrylate (for example, hexafluoroisopropyl (meth)acrylate), hexadecyl (meth)acrylate, hexadecenyl (meth)acrylate, octadecenyl (meth)acrylate, stearyl (meth)acrylate, and behenyl (meth)acrylate. Such polymerizable monomers may be used alone or in combination of two or more types.

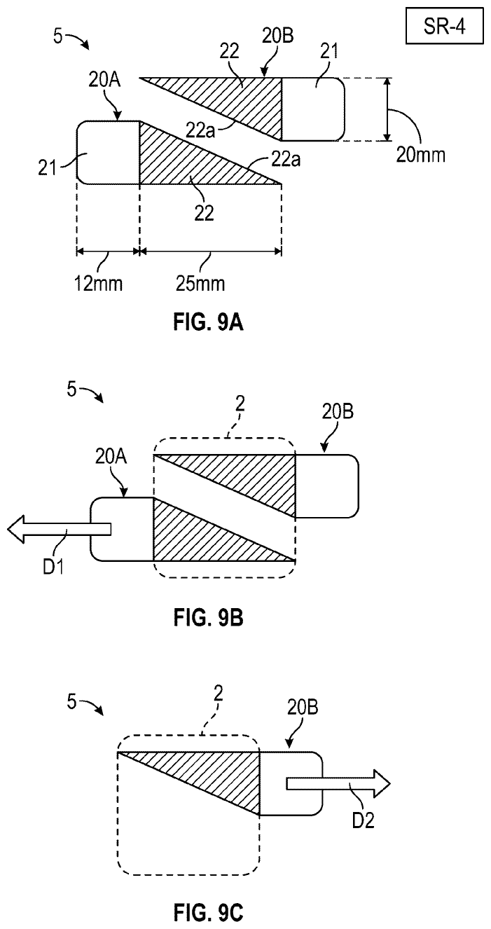

As illustrated in FIG. 1, the adhesive sheet unit 5 adheres to the second main surface 4b of the protective layer 4. The adhesive sheet unit 5 is formed so as to be stretchable in at least two directions when the article 2 is attached. The adhesive sheet unit 5 is disposed between the protective layer 4 formed on the adherend 3 and the article 2 when the article 2 is fixed on the adherend 3. The adhesive sheet unit 5 may be formed of one adhesive sheet, or may be formed by combining a plurality of adhesive sheets (for example, refer to FIGS. 9, 10, and 12). As illustrated in FIG. 2(a), the adhesive sheet unit 5 includes a stretchable substrate 13, a first adhesive layer 14 for adhering to the second main surface 4b, and a third adhesive layer 16 for adhering to the article 2. The first adhesive layer 14 is formed on the protective layer 4 side of the substrate 13. The third adhesive layer 16 is formed on the article 2 side of the substrate 13.

As the adhesive sheet unit 5 illustrated in FIG. 2(a), a stretchable double-sided tape may be employed. The substrate 13 is formed of, for example, a foam, and may be formed of polymer foam. Examples of the polymer foam include polyurethane foam, polyolefin foam (for example, polyethylene foam and polypropylene foam), and acrylic foam. The adhesive layers 14 and 16 are formed of, for example, a pressure sensitive adhesive (PSA).

As the adhesive sheet unit 5, a multilayer film that can be used as an elastic substrate may be employed as the substrate 13. The substrate 13 may have a low elastic modulus to minimize the forces required for elongation and tension. The substrate 13 may have low extensibility in order to minimize the distance required for elongation and tension. Preferably, most of the deformation of the substrate 13 is plastic deformation and the elastic deformation is minimal. To enable plastic deformation, the substrate 13 may have a core and at least a first skin on one side of the core. The core may have a first skin on one side and a second skin on the other side. A multilayer film designed for maximum strength includes an elastic layer forming a core sandwiched between two plastic deformation layers forming a skin.

As illustrated in FIG. 2(b), the adhesive sheet unit 5 may be a stretch peeling transfer tape formed only of the first adhesive layer 14 without the substrate 13. In the first adhesive layer 14 in this case, since the adhesive layer itself has appropriate stretchability and cohesive force even without the substrate 13, stretching and peeling of the adhesive layer alone can be performed.

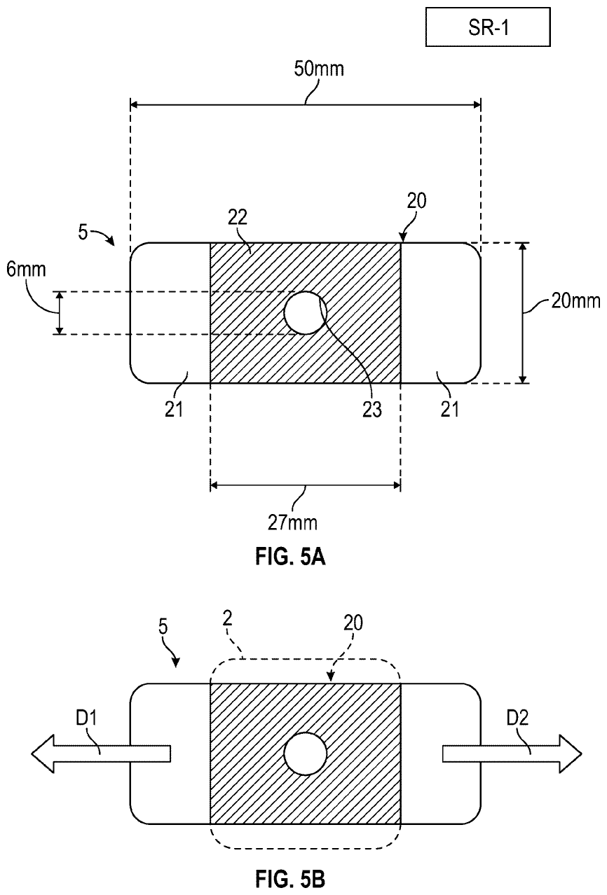

An example of the adhesive sheet unit 5 will be described with reference to FIG. 5. The adhesive sheet unit 5 of FIG. 5(a) is formed of one adhesive sheet 20. Note that the dimensions illustrated in FIG. 5(a) are merely examples, and can be changed as appropriate. The adhesive sheet unit 5 has a rectangular shape having a longitudinal direction. The adhesive sheet unit 5 is formed so as to be stretchable in at least two directions when the article 2 is attached. Here, the adhesive sheet unit 5 is formed to be stretchable in two directions of a direction D1 on one side and a direction D2 on the other side in the longitudinal direction (refer to FIG. 5(b)).

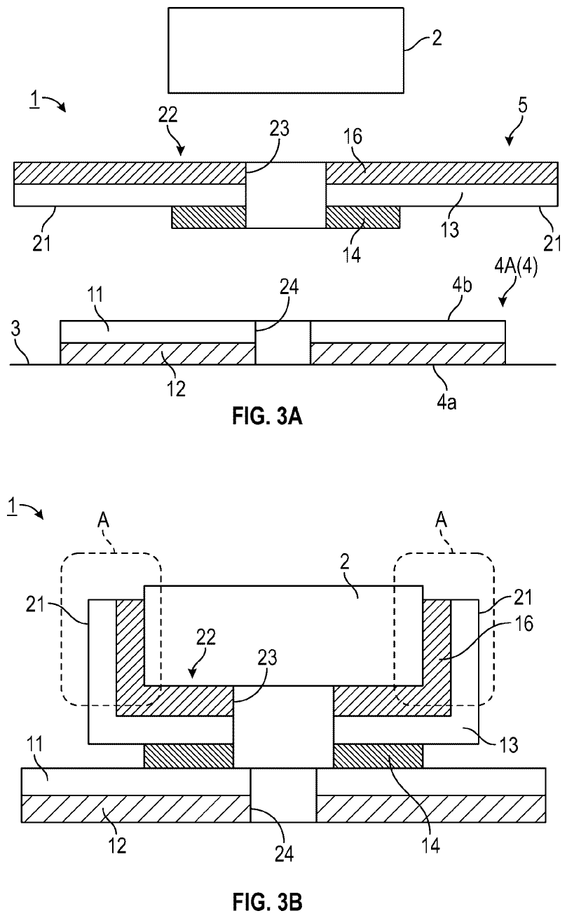

The adhesive sheet unit 5 includes a plurality of tab portions 21 that are not bonded to at least one of the protective layer 4 and the article 2 when the article 2 is attached. At least two of the plurality of tab portions 21 are formed at positions facing each other with the attachment position of the article 2 interposed therebetween in the adhesive sheet unit 5. Here, two tab portions 21 are formed at both ends in the longitudinal direction so as to face each other with the attachment position of the article 2 interposed therebetween. In the adhesive sheet unit 5, a portion other than the tab portions 21 is formed as an adhesive portion 22 having an adhesive force. The tab portion 21 has no adhesive force. Specifically, in the form of FIG. 2(a), a portion of the substrate 13 where the adhesive layers 14 and 16 are not formed may be the tab portion 21. As a result, the tab portion 21 is not adhered to the protective layer 4 and the article 2, and thus can be easily gripped with a finger. As illustrated in FIG. 3(a), one surface of the tab portion 21 may have an adhesive force so that temporary fixing to one of the protective layer 4A and the article 2 can be performed. In the example of FIG. 3(a), the tab portion 21 of the adhesive sheet unit 5 can be temporarily fixed to the article 2 as illustrated in FIG. 3 (b) (refer to the portion “A” in the drawing). With such a configuration, it is possible to reduce a feeling that the tab portion 21 becomes an obstacle during the attachment. At the time of peeling, a temporarily fixed portion (broken line portion) between the tab portion 21 and the article 2 is first peeled off, and thereafter, removal is performed in the same manner as in other forms. In the case of the example of FIG. 2(b), in order to form the tab portion 21, the coating layer 17 is formed so that the adhesive force of a part of the first adhesive layer 14 is not exerted. The coating layer 17 may have any configuration such as a film.

When it is preferable that the article 2 is in direct contact with or close to the adherend 3 (for example, human skin), a hole 23 may be formed in the adhesive sheet unit 5. A hole 24 may also be formed in the protective layer 4A (refer to FIG. 2(a)). For example, by forming the hole 24 of the protective layer 4A to be smaller than the hole 23 of the adhesive sheet unit 5, the first adhesive layer 14 can be prevented from directly adhering to the adherend 3. With such a configuration, the adherend 3 can be prevented from being damaged when the adhesive sheet unit 5 is peeled off.

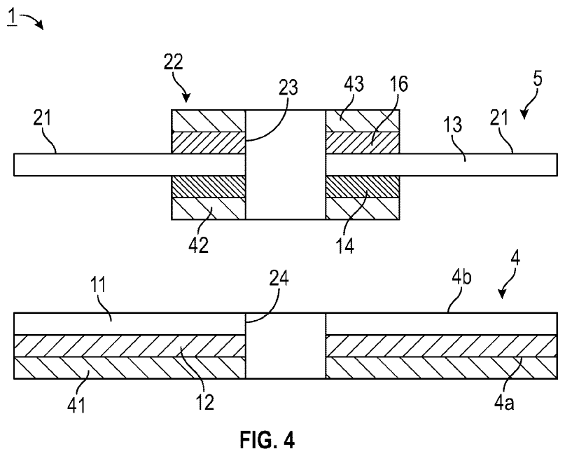

Note that FIG. 4 illustrates an example of a configuration before the article fixture 1 of FIG. 2(a) is attached. The article fixture 1 further includes peeling layers 41, 42, and 43. A peeling treatment agent is applied to a surface of the peeling layers 41, 42, and 43 facing the adhesive layers 12, 14, and 16. The peeling layer 41 is provided on the first main surface 4a side of the second adhesive layer 12. The peeling layer 42 is provided on the first adhesive layer 14, and the peeling layer 43 is provided so as to cover the third adhesive layer 16. At the time of attachment, a user peels off the peeling layer 41 and attaches the protective layer 4 to the adherend 3. Next, the peeling layer 42 is peeled off, and the first adhesive layer 14 is attached onto the second main surface 4b. At this time, it is preferable that positioning is performed so that the positions of the hole 23 and the hole 24 are not deviate from each other, and then the bonding is performed. The diameter of the hole 23 is preferably larger than that of the hole 24 so that the first adhesive layer 14 is not directly attached to the skin due to misalignment. After the adhesive portion 22 is attached, the peeling layer 43 is peeled off, and the device is bonded to the third adhesive layer 16. The third adhesive layer 16 may be provided on the adhesive portion 22 side, or may be provided on the device side and attached onto the substrate 13.

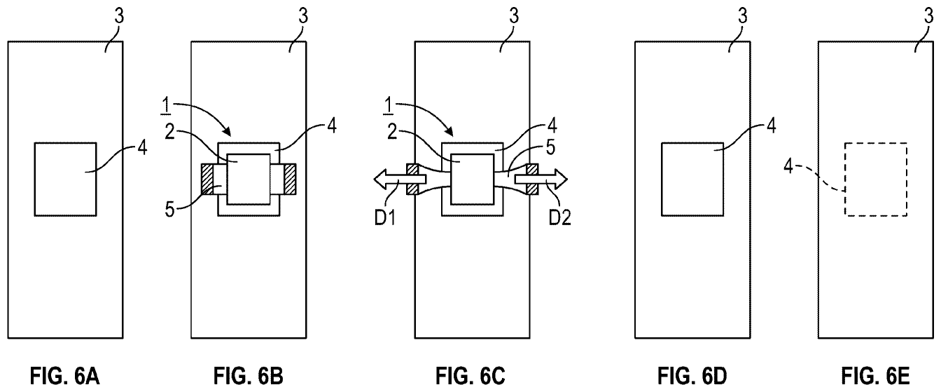

Next, with reference to FIG. 6, a method for fixing the article 2 by the article fixture 1 and a method for removing the article 2 fixed by the article fixture 1 will be described. As illustrated in FIG. 6(a), first, the protective layer 4A is formed on the adherend 3. Next, as illustrated in FIG. 6(b), the adhesive sheet unit 5 is attached onto the protective layer 4B, and the article 2 is further attached thereon. Accordingly, the fixing of the article 2 is completed.

When the article 2 is removed, as illustrated in FIG. 6(c), the adhesive sheet unit 5 is stretched in two directions D1 and D2 while the protective layer 4 is left on the adherend 3. Thereby, as illustrated in FIG. 6(d), the adhesive sheet unit 5 is detached from the protective layer 4 together with the article 2. Next, as illustrated in FIG. 6(e), the protective layer 4 is peeled off from the adherend 3. A new article 2 may be fixed without peeling off the protective layer 4.

Next, actions and effects of the article fixture 1, the method for removing the article 2, and the adhesive sheet unit 5 according to the present embodiment will be described.

As wearable devices in the medical field and the like, various devices such as a continuous blood glucose measurement sensor, an insulin pump, a heart rate sensor, and a thermometer are used for health management and physical condition management. Then, mounting the device in a stable skin contact and a stable position is an important factor for obtaining reliable data. In the related art, medical tapes are commonly used for such fixation. When the wearable device is fixed with the medical tape, there is a problem in that the skin is damaged when the medical tape is removed. Generally, the medical tape should be removed with a slow peeling to minimize damage to the skin upon removal. However, there is a problem that damage to the skin increases when the medical tape is peeled off from the skin together with the hard device. Further, since the wearable device is repeatedly worn on the same place, there is a problem that damage is accumulated on the skin.

On the other hand, an article fixture 1 according to the present embodiment is the article fixture 1 for fixing an article 2 on an adherend 3, the article fixture 1 including: a protective layer 4 configured to be formed on the adherend 3, the protective layer including a first main surface 4a facing the adherend 3 and a second main surface 4b opposite to the adherend 3; and an adhesive sheet unit 5 configured to bond to the second main surface 4b of the protective layer 4, wherein the adhesive sheet unit 5 includes a first adhesive layer 14 configured to bond to the second main surface 4b, and the adhesive sheet unit 5 is formed to be stretchable in at least two directions when the article 2 is attached.

In this case, the article 2 is fixed to the adherend 3 with the adhesive sheet unit 5 and the protective layer 4 interposed therebetween. When the article 2 is removed from the adherend 3, the adhesive sheet unit 5 can be detached from the protective layer 4 by being stretched in at least two directions with the protective layer 4 left on the adherend 3. When the adhesive sheet unit 5 is detached, the article 2 is also removed from the protective layer 4. At this time, since the adhesive sheet unit 5 is removed in a state where the adherend 3 is covered with the protective layer 4, damage to the adherend 3 is reduced. Further, since the adhesive sheet unit 5 is peeled off while being stretched in a direction parallel to the surface of the adherend 3, damage to the adherend 3 can be reduced, and the protective layer 4 can be suppressed from being peeled off. After the article 2 is removed, only the protective layer 4 may be peeled off from the adherend 3, so that damage to the adherend 3 can be reduced as compared with the case where the article 2 and the protective layer 4 are simultaneously peeled off. Alternatively, the protective layer 4 may be left even after the article 2 is removed, and the protective layer 4 may be used when a new article 2 is fixed. In this case, since the frequency of replacement of the protective layer 4 can be reduced, damage to the adherend 3 due to repeated peeling of the protective layer 4 can be reduced. In addition, when the adherend 3 is soft such as skin, if the pressure-sensitive adhesive sheet unit 5 is formed to stretch in one direction, the adherend 3 may also stretch in one direction along with the stretching, and damage may occur or the adherend 3 may not be successfully peeled off.

The adhesive sheet unit 5 may have a plurality of tab portions 21 that are not bonded to at least one of the protective layer 4 and the article 2 when the article 2 is attached. In this case, the operator can easily grasp the tab portion 21 with his/her fingers and stretch the adhesive sheet unit 5.

At least two of the plurality of tab portions 21 may be formed at positions facing each other with the attachment position of the article 2 interposed therebetween in the adhesive sheet unit 5. In this case, since the adhesive sheet unit 5 can be pulled from both sides across the article 2, the removal operation can be easily performed, and the way of applying a force to the adhesive sheet unit 5 can be stabilized. The protective layer 4 may include a protective substrate 11 having a second main surface 4b and a second adhesive layer 12 formed on the first main surface 4a. In this case, the operator can hold the protective substrate 11 and easily attach the protective layer 4 to the adherend 3.

The second adhesive layer 12 of the protective layer 4 may have a holding force of 50 minutes or more against Protein Leather (trade name) under load conditions of 30° C., 75% RH, and 300 g. In this case, the protective layer 4 can be attached to the adherend 3 for a long period of time in a state where a load on the adherend 3 is small.

The adhesive sheet unit 5 and the protective layer 4 are respectively formed with holes 23 and 24, and the hole 24 of the protective layer 4 may be smaller than the hole 23 of the adhesive sheet unit 5. In this case, a needle or the like for injecting medicine can pass through the holes 24 and 23. Further, the first adhesive layer 14 of the adhesive sheet unit 5 can be prevented from protruding from the hole 24 of the protective layer 4 and being attached to the adherend 3.

The protective substrate 11 of the protective layer 4 may have a tensile strength of less than 100 N/25 mm. In this case, the protective layer 4 can be attached to the adherend 3 for a long period of time in a state where a load on the adherend 3 is small.

The protective layer 4 may be formed as a layer by applying a protective liquid to the adherend 3. In this case, the protective layer 4 having a desired shape can be formed.

A method for removing an article according to the present embodiment is a method for removing the article 2 fixed by the above-described article fixture 1, the method including detaching the adhesive sheet unit 5 from the protective layer 4 by stretching the adhesive sheet unit 5 in at least two directions with the protective layer 4 left on the adherend 3.

According to the method for removing an article, the same actions and effects as those of the article fixture 1 described above can be obtained.

An adhesive sheet unit 5 according to an embodiment of the present invention is disposed between a protective layer 4 formed on an adherend 3 and an article 2 when the article 2 is fixed on the adherend 3, the adhesive sheet unit 5 including a first adhesive layer 14 bonded to the protective layer 4, wherein the adhesive sheet unit 5 is formed to be stretchable in at least two directions when the article 2 is attached.

According to the adhesive sheet unit 5, the same actions and effects as those of the article fixture 1 described above can be obtained.

The present invention is not intended to be limited to the embodiments described above.

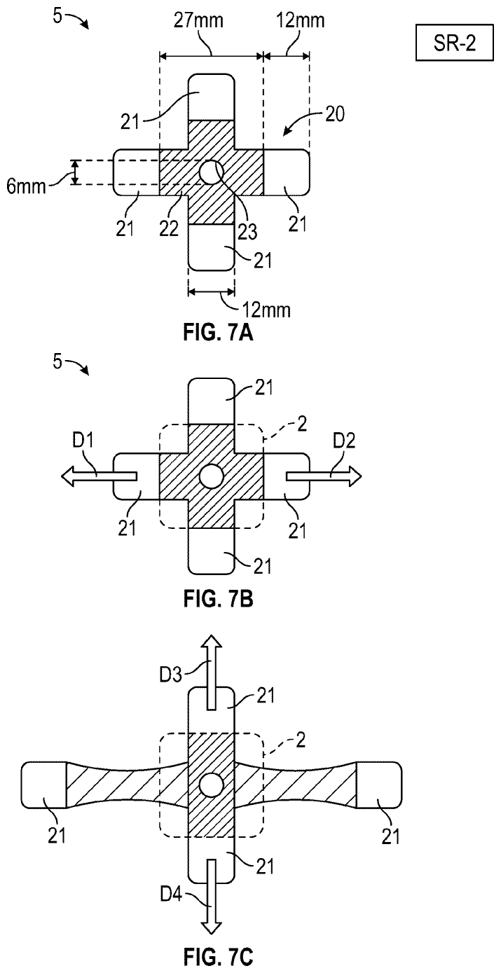

The configuration of the adhesive sheet unit 5 is not particularly limited. For example, the adhesive sheet unit 5 illustrated in FIG. 7 may be employed. The adhesive sheet unit 5 illustrated in FIG. 7(a) has a cross shape. A total of four tab portions 21 are formed at four end portions so as to sandwich the position where the article 2 is attached in two directions. The adhesive sheet unit 5 can stretch in four directions (directions D1 to D4). When removing the adhesive sheet unit 5, as illustrated in FIG. 7(b), the adhesive sheet unit 5 is stretched in the direction D1 and the direction D2 opposite to each other. Next, as illustrated in FIG. 7(c), the adhesive sheet unit 5 is stretched in a direction D3 and a direction D4 that are orthogonal to the directions D1 and D2 and face each other.

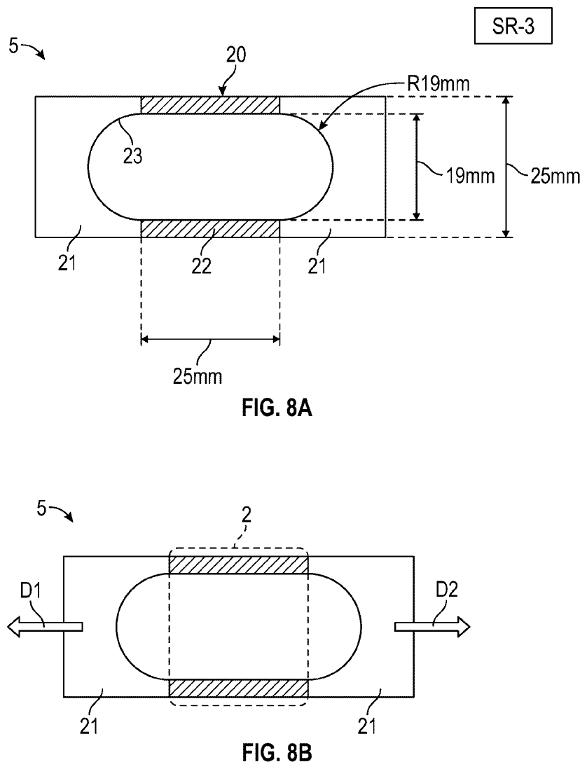

The adhesive sheet unit 5 illustrated in FIG. 8 may be employed. The adhesive sheet unit 5 illustrated in FIG. 8(a) is different from the adhesive sheet unit 5 illustrated in FIG. 5(a) in that the former includes a large oval hole 23. The hole 23 is an oval extending in the longitudinal direction of the adhesive sheet unit 5 and reaches the tab portions 21 on both sides. The other configuration of the adhesive sheet unit 5 of FIG. 8 is the same as that of FIG. 5.

The adhesive sheet unit 5 illustrated in FIG. 9 may be employed. The adhesive sheet unit 5 illustrated in FIG. 9(a) is formed by a combination of two adhesive sheets 20A and 20B. The adhesive portion 22 of each of the adhesive sheets 20A and 20B has a right triangular shape. The adhesive sheets 20A and 20B are disposed so as to face each other in a state where the oblique sides 22a thereof are separated from each other. Accordingly, the adhesive sheet unit 5 is formed in a rectangular shape by a combination of the adhesive sheets 20A and 20B. The adhesive sheet unit 5 is formed to be stretchable in two directions of a direction D1 on one side and a direction D2 on the other side in the longitudinal direction. When removing the adhesive sheet unit 5, as illustrated in FIG. 9(b), the adhesive sheet 20A is stretched in the direction D1 and removed. Next, as illustrated in FIG. 9(c), the adhesive sheet 20B is stretched in the direction D2 and removed.

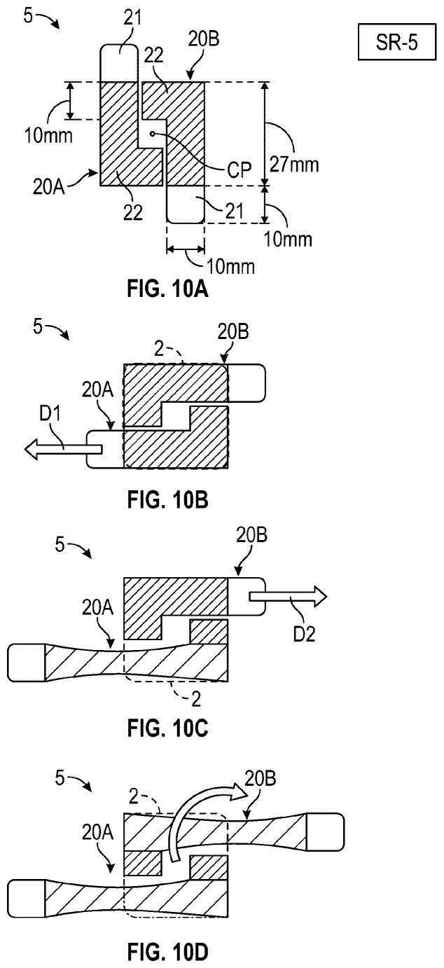

The adhesive sheet unit 5 illustrated in FIG. 10 may be employed. The adhesive sheet unit 5 illustrated in FIG. 10(a) is formed by a combination of two adhesive sheets 20A and 20B. The adhesive portion 22 of each of the adhesive sheets 20A and 20B has an L-shape. The adhesive sheets 20A and 20B are disposed so as to be point-symmetrical to each other with respect to a center point CP. The adhesive sheet unit 5 is formed to be stretchable in two directions of a direction D1 on one side and a direction D2 on the other side. When removing the adhesive sheet unit 5, as illustrated in FIG. 10(b), the adhesive sheet 20A is stretched in the direction D1. Next, as illustrated in FIG. 10(c), the adhesive sheet 20B is stretched in the direction D2. As illustrated in FIG. 10(d), since most of the regions of the adhesive sheets 20A and 20B are stretched and are in a state of being easily peeled off, the article 2 can be easily removed.

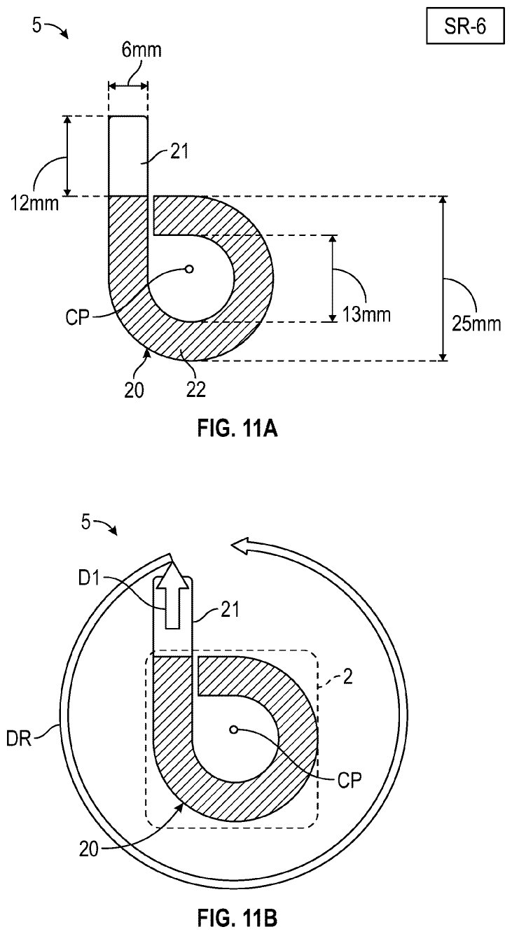

The adhesive sheet unit 5 illustrated in FIG. 11 may be employed. The adhesive sheet unit 5 illustrated in FIG. 11(a) is formed of one adhesive sheet 20. The adhesive sheet unit 5 has a shape in which a long adhesive sheet 20 is wound in a circle. The adhesive sheet unit 5 can be stretched in one direction D1 and can be stretched in a direction DR over the entire circumference. In the state of being able to be stretched in the direction DR, it is possible to be stretched in all directions with respect to the center point CP, and thus, the adhesive sheet unit 5 can be stretched in a plurality of directions. When removing the adhesive sheet unit 5, as illustrated in FIG. 11(b), the adhesive sheet unit 5 is stretched in the direction D1. Next, the tab portion 21 is rotated in the direction DR over the entire circumference while pulling the tab portion 21. Thus, the adhesive sheet unit 5 is gradually peeled off.

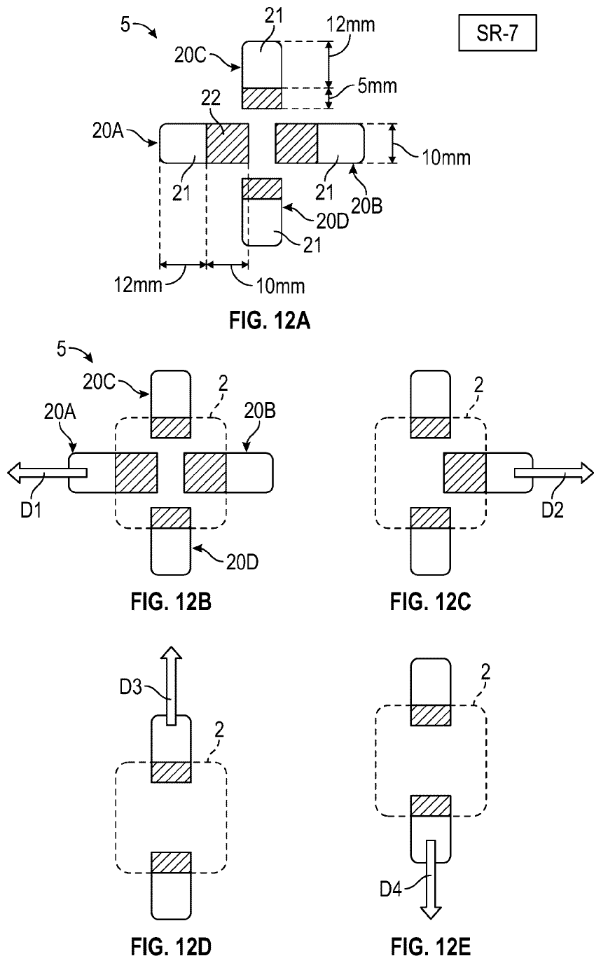

An adhesive sheet unit 5 illustrated in FIG. 12 may be employed. The adhesive sheet unit 5 illustrated in FIG. 12(a) has a cross shape by combining four adhesive sheets 20A, 20B, 20C, and 20D.

The adhesive sheets 20A, 20B, 20C, and 20D are disposed such that a total of four tab portions 21 are formed at four end portions of the cross shape so as to sandwich the position where the article 2 is attached in two directions. The adhesive sheet unit 5 can stretch in four directions (directions D1 to D4). When removing the adhesive sheet unit 5, as illustrated in FIG. 12(b), the adhesive sheet 20A is stretched in the direction D1 and removed. Next, as illustrated in FIG. 12(c), the adhesive sheet 20B is stretched in the direction D2 and removed. Next, as illustrated in FIG. 12(d), the adhesive sheet 20C is stretched in the direction D3 and removed. Next, as illustrated in FIG. 12(e), the adhesive sheet 20D is stretched in the direction D4 and removed.

### Evaluation Test

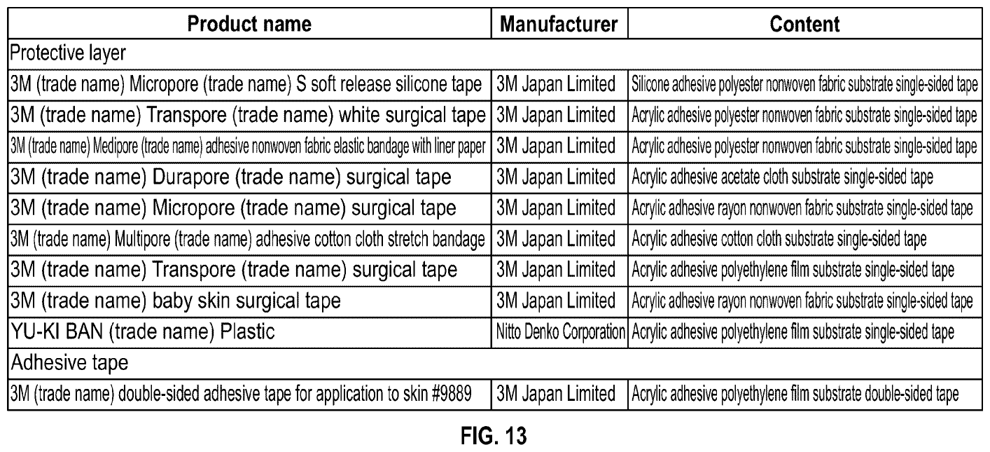

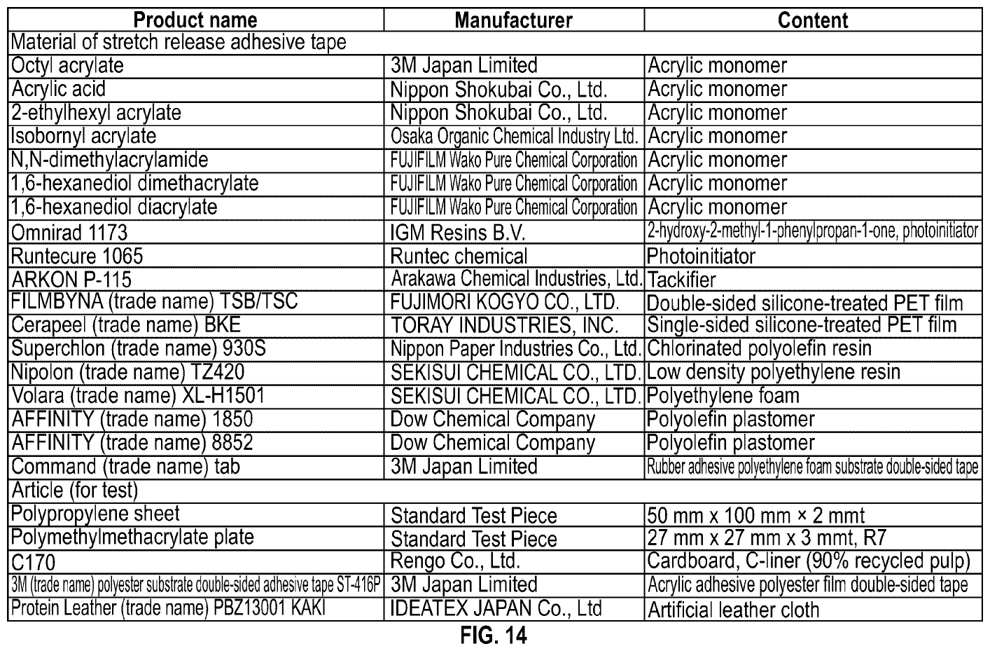

Various tests for evaluating the article fixture were performed. The members that can be selected for testing are shown in FIGS. 13 and 14. Three types of stretch release adhesive tapes “SRT-1”, “SRT-2”, and “SRT-3” were prepared for use in the test. The stretch release bonding tape is a tape used as an adhesive sheet unit in the embodiment.

### SRT-1: Double-Sided Tape of Polyolefin Film

First, a stretchable polyolefin film was prepared by co-extrusion of AFFINITY 1850 and AFFINITY 8852. A layered structure of the film is “8852/1850/8852=0.8 mil/0.4 mil/0.8 mil”. Next, an adhesive solution in which isooctyl acrylate and acrylic acid were copolymerized in a solution was prepared, and the adhesive solution was applied onto FILMBYNA (trade name) TSB/TSC using a knife coater and dried (60° C. for 2 minutes+120° C. for 2 minutes). The resulting PSA sheet had a thickness of about 20 μm. The obtained adhesive transfer tape was stacked on both surfaces of the stretchable polyolefin film after corona treatment to obtain “SRT-1” as a polyolefin film DC tape.

### SRT-2: Thick Acrylic Adhesive Transfer Tape

A mixture of “octyl acrylate/Omnirad 1173=100/0.02 (parts by mass)” was prepared and filled in a glass container. The dissolved oxygen in the mixture was replaced with nitrogen gas. Thereafter, partial polymerization was performed by ultraviolet irradiation for several minutes using a low-pressure mercury lamp to obtain a viscous liquid having a viscosity of about 5000 MPa·s. To the viscous liquid, 2 parts of acrylic acid and 0.15 parts of Omnirad 1173 were added and mixed with a Thinky mixer ARM-310. The resulting mixture was coated on FILMBYNA (trade name) TSB/TSC at a thickness of 500 μm. Subsequently, in order to block oxygen that inhibits polymerization, Cerapeel (trade name) BKE was placed on the coating surfaces and irradiated using a low pressure mercury lamp (1 mW/cm² for 3 minutes and 5 mW/cm² for 3 minutes). As a result, “SRT-2” was obtained as a thick acrylic adhesive transfer tape.

### SRT-3: Foamed Polyethylene Double-Coated Tape

A mixture of “2-ethylhexyl acrylate/isobornyl acrylate/1,6-hexanediol dimethacrylate/Runtecure 1065=56.2/18 7/3 1/0.03/0.03 (parts by mass)” was prepared and filled into a glass container. The dissolved oxygen in the mixture was replaced with nitrogen gas. Thereafter, partial polymerization was performed by ultraviolet irradiation for several minutes using a low-pressure mercury lamp to obtain a viscous liquid having a viscosity of about 3000 MPa·s. To the viscous liquid, 15 parts of IBOA, 1.8 parts of AA, 3 parts of DMAA, 0.48 parts of Runtecure 1065, 2 parts of ARKON P-115, and 0.4 parts of Superchlon (trade name) 930S were added, and mixed with a Thinky mixer ARM-310. The resulting mixture was coated on FILMBYNA (trade name) TSB/TSC at a thickness of 100 μm. Thereafter, in order to block oxygen that inhibits polymerization, Cerapeel (trade name) BKE was placed on the coating surfaces and irradiated using a low pressure mercury lamp (0.5 mW/cm² for 3 minutes and 70 mW/cm² for 3 minutes) to obtain an adhesive transfer tape. Next, Nipolon (trade name) TZ420 was extruded and stacked to a thickness of 10 to 20 μm on both surfaces of a foamed polyethylene sheet Volara (trade name) XL-H1501, a corona treatment was applied to the re-surface, and then the adhesive transfer tape obtained above was laminated to obtain SRT-3.

### Adhesive Force Between Peeling and Tension

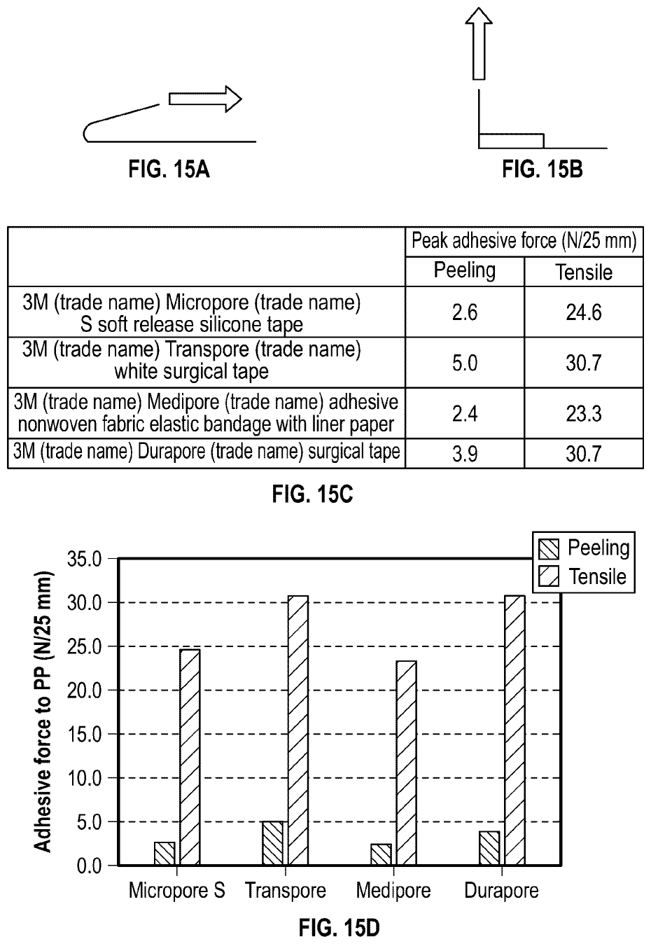

In order to evaluate the difference in stress on the skin when the protective layer tape was removed, the adhesive force of the protective layer tape was compared between a peeling mode and a tension mode. The peeling mode test assumes protective layer tape removal after removal of the article by the present patent method. A sample having a width of 25 mm was prepared and stacked on a PP plate using a 2 kg roller (50 mm/sec in reciprocation). The tape was peeled off in the 180° direction using TENSILON at a test speed of 300 mm/min (refer to FIG. 15(a)). On the other hand, the tensile mode test assumes that the protective layer tape is removed together with the article by a known method. A sample having a width of 25 mm was prepared and stacked on a PP plate using a 2 kg roller (50 mm/sec in reciprocation). A PMMA plate was stacked on the back surface of the stacked sample with #9889 (Double-sided tape for skin sticking, manufactured by 3M Company). The stacked sample was pulled in a 90° direction at a test speed of 300 mm/min using TENSILON (A&D Company, Limited) (refer to FIG. 15(b)).

The results of the above tests are illustrated in FIGS. 15(c) and (d). The adhesive force in the tensile mode was about 10 times the adhesive force in the peeling mode. As described above, it can be seen that a large stress is applied to the skin in the known method, whereas the stress can be significantly reduced in the present patent method. The stress reduction suggests reduction in pain at the time of peeling and reduction in risk of skin laceration and the like.

Next, in order to compare the skin peeling risk at the time of removing the protective layer tape, a peeling test using a cardboard was performed. First, a cardboard test substrate was prepared. The C170 was cut to a size of 50 mm×100 mm and a half cut grid of a size of 3 mm×3 mm was provided by a pinnacle die. The depth of cut was 60% of the total thickness. The cut C170 was stacked to a 2 mm thick PP plate using ST-416 (thin double-sided tape, manufactured by 3M Company) to give a cardboard test substrate.

Next, as a peeling mode test, a 25 mm-wide protective layer tape sample was stacked on a cardboard test substrate using a 2 kg roller, and then peeled off at a speed of 300 mm/min in a direction of 180 degrees. The number of damaged grids having an area of 25 mm×25 mm at this time was counted. As a tensile mode test, a 25 mm-wide tape sample was stacked to a cardboard test substrate using a 2 kg roller and then a PMMA plate was stacked to the back surface of the #9889 stacked sample. The stacked tape samples were pulled in a 90 degree direction at a test speed of 300 mm/min. The number of damaged grids having an area of 25 mm×25 mm at this time was counted.

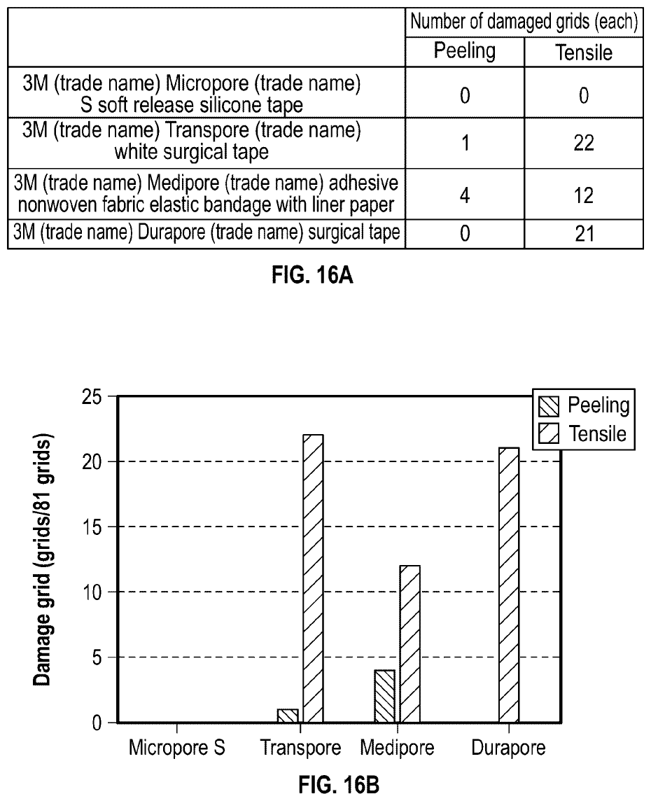

The results of the above tests are illustrated in FIGS. 16(a) and (c). It was confirmed that there was a difference in damage between the peeling mode and the tension mode of the peeling layer tape, and many grids were removed in the tension mode. It is understood that this has a high risk of removing cells of the skin, tearing of the skin, peeling of the skin, and the like.

### Comparative Test

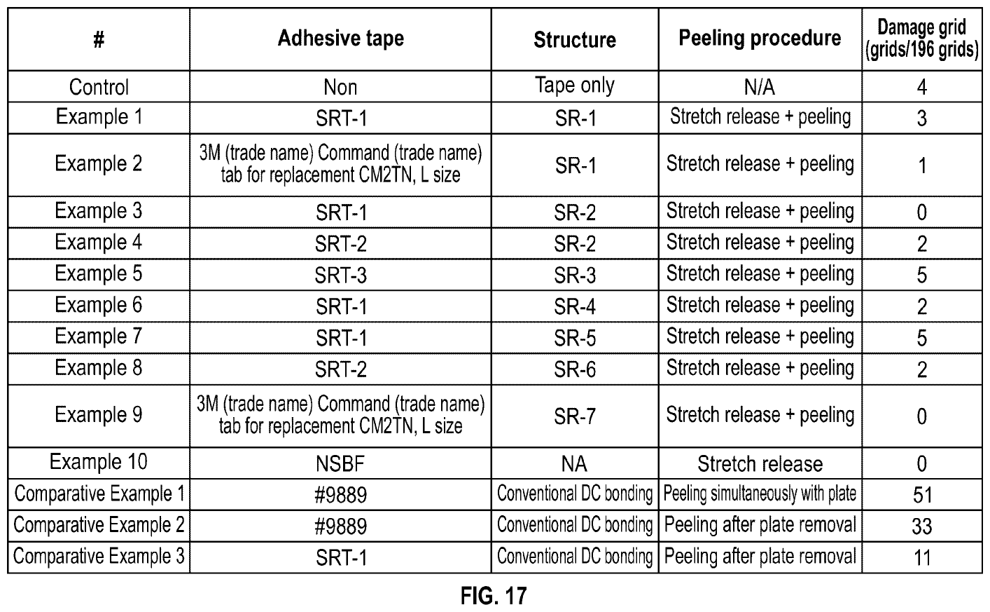

Next, as illustrated in FIG. 17, examples and comparative examples were prepared, and how much skin damage could be suppressed by the article fixture of the example was evaluated. First, Medipore (trade name) having a width of 40 mm (bonding area of 40 mm×40 mm, with a tab having a length of 25 mm) was stacked as a protective layer tape for a protective layer on the cardboard test substrate described in the grid test using the above-described cardboard, and was well pressed with a finger. Each adhesive tape was then stacked onto the back surface of the PMMA plate. Thereafter, it was placed on Medipore (trade name) and pressed well with a finger. After a few minutes, the PMMA plate, adhesive tape, and Medipore (trade name) were removed according to the respective procedures. Finally, the number of broken grids was counted. Hereinafter, test methods of Examples and Comparative Examples will be described.

### EXAMPLES

In Examples 1 to 9, any one of the above-mentioned “SRT-1”, “SRT-2”, “SRT-3”, and Command tab (trade name, manufactured by 3M Company) was adopted as a stretch release adhesive tape corresponding to the adhesive sheet unit. As the structure of the adhesive sheet unit, any one of “SR-1” illustrated in FIG. 5, “SR-2” illustrated in FIG. 7, “SR-3” illustrated in FIG. 8, “SR-4” illustrated in FIG. 9, “SR-5” illustrated in FIG. 10, “SR-6” illustrated in FIG. 11, and “SR-7” illustrated in FIG. 12 was employed. The adhesive tapes and structures used in Examples 1 to 9 are illustrated in FIG. 17. In

Examples 1 to 9, each adhesive tape was stretched in a defined direction (refer to FIGS. 5 and 7 to 12) and removed from the Medipore (trade name) along with the PMMA plate. The Medipore (trade name) was then slowly peeled from the cardboard test substrate while being folded back in a 180° direction. In Example 10, NSBF was applied to a cardboard test substrate and dried, “SRT-1” of “SR-1” was attached thereto, and a PMMA plate was further attached thereto and subjected to pressure bonding. Thereafter, “SRT-1” was removed by stretch release, and damage to the cardboard test substrate was confirmed.

### Comparative Examples

In Comparative Examples 1 and 2, an adhesive tape #9889 was attached onto the protective tape, and a PMMA plate was attached onto the adhesive tape. In Comparative Example 1, the protective layer tape was peeled off together with the PMMA plate and the adhesive tape from the cardboard test substrate. In Comparative Example 2, the PMMA plate was peeled off from the Medipore (trade name) together with the adhesive tape, and then the Medipore (trade name) was slowly peeled off from the cardboard test substrate while being folded in a 180° direction. In Comparative Example 3, “SRT-1” was attached as an adhesive tape, and a PMMA plate was attached onto the adhesive tape. In Comparative Example 3, the PMMA plate was peeled off from the Medipore (trade name) without stretch release of

SRT-1, and then the Medipore (trade name) was slowly peeled off from the cardboard test substrate while being folded in a 180° direction.

The test results are shown in the item of the damage grid in FIG. 17. In Comparative Example 1, a known removal method is assumed, and it is estimated that the skin is greatly damaged by removing the tape integrated with the article. In Comparative Examples 2 and 3, a part of the present patent method is employed, and the protective layer tape is peeled off after the article is removed. However, it was confirmed that the removal of the article is performed by not stretch-releasing but forcibly pulling, and thus there is a risk that the skin is damaged through the protective layer tape. On the other hand, in all of Examples 1 to 10, it was confirmed that damage can be reduced as compared with Comparative Example.

### Static Shear Test

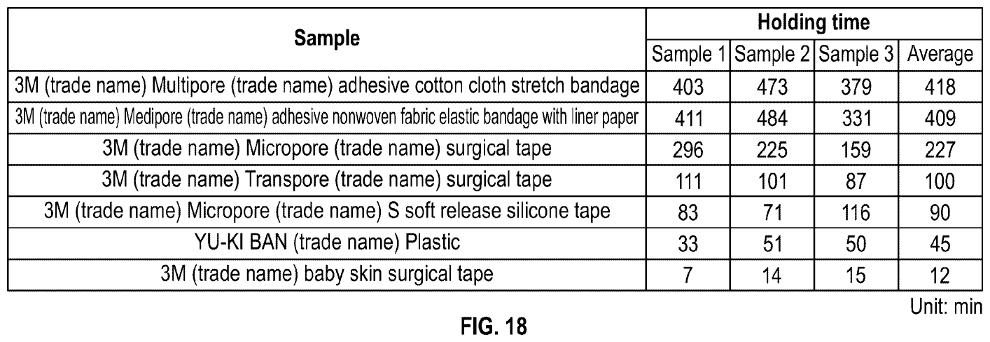

A holding power test using Protein Leather (trade name) PBZ13001 KAKI (IDEATEX JAPAN Co., Ltd) will be described with reference to FIG. 18. A sample tape was prepared with a size of 25 mm×75 mm, leaving an adhesive area of 25 mm×25 mm at a tip, and a 25 μm thick polyester (PET) film was laminated to the 25 mm×50 mm area to prevent stretch peeling of the tape sample. Protein Leather (trade name) having a size of 30 mm×125 mm was prepared as a test substrate. A PET film stacked tape sample was stacked on the end of Protein Leather (trade name), and the stacked sample was prepared by pressurizing with a 2 kg roller.

Protein Leather (trade name) is made of protein powder and special resin. Protein Leather (trade name) has the skin of a person who prefers excellent texture, and is used for cosmetic test applications by many cosmetic manufacturers. The surface layer is made of a urethane material and a protein powder, and has the same moisture absorbing and releasing performance as the natural material.

Next, the stacked sample was subjected to a holding power test using a 145-DP RETENTIVITY TESTER (YASUDA SEIKI SEISAKUSHO, LTD.). The stacked sample was held in a test environment of 30 deg C and 75% RH for 1 hour without a weight load, then each sample was loaded with a weight of 300 g, and the holding time until the tape material dropped was measured. Measurement results are illustrated in FIG. 18.

### Tensile Strength Test

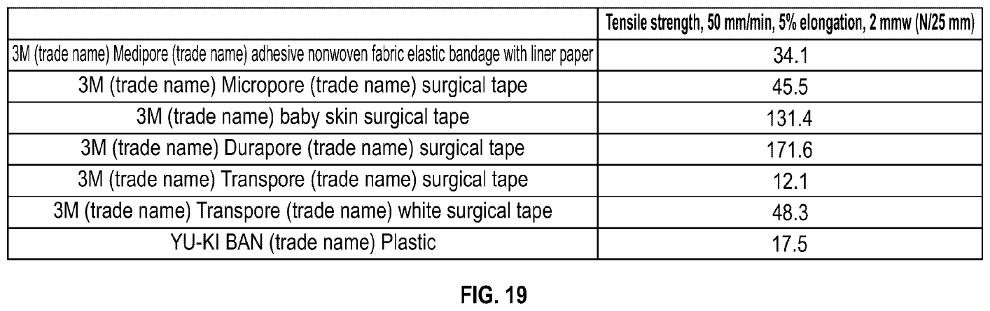

The tensile strength test will be described with reference to FIG. 19. Here, the sample size was 25 mm wide×100 mm or more. A distance between chucks was set to 50 mm, a tensile test was performed at a speed of 50 mm/min, and a tensile strength at a 5% tensile point (2.5 mm) was measured. When the tensile strength is high, stress applied to the skin increases, which may cause skin problems. Test results are illustrated in FIG. 19. Here, a condition of less than 100 N/25 mm is a favorable condition.

### Aspect 1

An article fixture for fixing an article on an adherend, the article fixture including: a protective layer configured to be formed on the adherend, the protective layer including a first main surface facing the adherend, and a second main surface opposite to the adherend; and an adhesive sheet unit configured to bond to the second main surface of the protective layer, wherein the adhesive sheet unit includes a first adhesive layer configured to bond to the second main surface, and the adhesive sheet unit is formed to be stretchable in at least two directions when the article is attached.

### Aspect 2

The article fixture according to aspect 1, wherein the adhesive sheet unit includes a plurality of tab portions that are not bonded to at least one of the protective layer and the article when the article is attached.

### Aspect 3

The article fixture according to aspect 2, wherein at least two of the plurality of tab portions are formed at positions facing each other with an attachment position of the article interposed between the at least two of the plurality of tab portions in the adhesive sheet unit.

### Aspect 4

The article fixture according to any one of aspects 1 to 3, wherein the protective layer includes a protective substrate including the second main surface, and a second adhesive layer formed on the first main surface.

### Aspect 5

The article fixture according to aspect 4, wherein the second adhesive layer of the protective layer has a holding force of 50 minutes or more against Protein Leather under load conditions of 30° C., 75% RH, and 300 g.

### Aspect 6

The article fixture according to any one of aspects 1 to 5, wherein a hole is formed in each of the adhesive sheet unit and the protective layer, and the hole of the protective layer is smaller than the hole of the adhesive sheet unit.

### Aspect 7

The article fixture according to any one of aspects 1 to 6, wherein the protective substrate of the protective layer has a tensile strength of less than 100 N/25 mm.

### Aspect 8

The article fixture according to any one of aspects 1 to 7, wherein the protective layer is formed as a layer by applying a protective liquid to the adherend.

### Aspect 9

A method for removing the article fixed by the article fixture described in any one of aspects 1to 8, the method including detaching the adhesive sheet unit from the protective layer by stretching the adhesive sheet unit in at least two directions with the protective layer left on the adherend.

### Aspect 10

An adhesive sheet unit disposed between a protective layer formed on an adherend and an article when the article is fixed on the adherend, the adhesive sheet unit including a first adhesive layer bonded to the protective layer, wherein the adhesive sheet unit is formed to be stretchable in at least two directions when the article is attached.

1. An article fixture for fixing an article on an adherend, the article fixture comprising:
a protective layer configured to be formed on the adherend, the protective layer including
a first main surface facing the adherend, and
a second main surface opposite to the adherend; and
an adhesive sheet unit configured to bond to the second main surface of the protective layer,
wherein the adhesive sheet unit includes a first adhesive layer configured to bond to the second main surface, and
the adhesive sheet unit is formed to be stretchable in at least two directions when the article is attached.

2. The article fixture according to claim 1, wherein the adhesive sheet unit includes a plurality of tab portions that are not bonded to at least one of the protective layer and the article when the article is attached.

3. The article fixture according to claim 2, wherein at least two of the plurality of tab portions are formed at positions facing each other with an attachment position of the article interposed between the at least two of the plurality of tab portions in the adhesive sheet unit.

4. The article fixture according to claim 1, wherein the protective layer includes
a protective substrate including the second main surface, and
a second adhesive layer formed on the first main surface.

5. The article fixture according to claim 4, wherein the second adhesive layer of the protective layer has a holding force of 50 minutes or more against Protein Leather under load conditions of 30° C., 75% RH, and 300 g.

6. The article fixture according to claim 1, wherein
a hole is formed in each of the adhesive sheet unit and the protective layer, and
the hole of the protective layer is smaller than the hole of the adhesive sheet unit.

7. The article fixture according to claim 1, wherein the protective substrate of the protective layer has a tensile strength of less than 100 N/25 mm.

8. The article fixture according to claim 1, wherein the protective layer is formed as a layer by applying a protective liquid to the adherend.

9. A method for removing the article fixed by the article fixture described in claim 1, the method comprising
detaching the adhesive sheet unit from the protective layer by stretching the adhesive sheet unit in at least two directions with the protective layer left on the adherend.

10. An adhesive sheet unit disposed between a protective layer formed on an adherend and an article when the article is fixed on the adherend, the adhesive sheet unit comprising
a first adhesive layer bonded to the protective layer, wherein the adhesive sheet unit is formed to be stretchable in at least two directions when the article is attached.

11. The article fixture according to claim 10, wherein the adhesive sheet unit includes a plurality of tab portions that are not bonded to at least one of the protective layer and the article when the article is attached.

12. The article fixture according to claim 11, wherein at least two of the plurality of tab portions are formed at positions facing each other with an attachment position of the article interposed between the at least two of the plurality of tab portions in the adhesive sheet unit.

13. The article fixture according to claim 10, wherein the protective layer includes
a protective substrate including the second main surface, and
a second adhesive layer formed on the first main surface.

14. The article fixture according to claim 13, wherein the second adhesive layer of the protective layer has a holding force of 50 minutes or more against Protein Leather under load conditions of 30° C., 75% RH, and 300 g.

15. The article fixture according to claim 10, wherein
a hole is formed in each of the adhesive sheet unit and the protective layer, and
the hole of the protective layer is smaller than the hole of the adhesive sheet unit.

16. The article fixture according to claim 10, wherein the protective substrate of the protective layer has a tensile strength of less than 100 N/25 mm.
